(19) United States
(12) Patent Application Publication
(10) Pub. No.: US 2022/0039733 A1
(43) Pub. Date: Feb. 10, 2022

(54) Method to Detect Perfusion and Brain Functional Activities Using Hyperpolarized 129Xe MRI

(71) Applicant: LAKEHEAD UNIVERSITY, Thunder Bay (CA)

(72) Inventors: Mitchell Albert, Thunder Bay (CA); Francis Hane, Thunder Bay (CA); Yurii Shepelytskyi, Thunder Bay (CA); Tao Li, Thunder Bay (CA); Vira Grynko, Thunder Bay (CA)

(21) Appl. No.: 17/396,272

(22) Filed: Aug. 6, 2021

Related U.S. Application Data
(60) Provisional application No. 63/062,640, filed on Aug. 7, 2020.

(51) Int. Cl. A61B 5/00; A61B 5/026; A61B 5/055; G01R 33/48; G01R 33/563; G01R 33/56

(52) U.S. Cl. CPC A61B 5/4064; A61B 5/0048; A61B 5/0263; A61B 5/055; G01R 33/5601; A61B 5/0044; G01R 33/4806; G01R 33/56366; A61B 5/0042

(57) ABSTRACT

Described herein is a method to detect perfusion and brain functional activity using Hyperpolarized xenon-129 (¹²⁹Xe) Time-of-Flight (TOF) Magnetic Resonance Imaging (MRI). Specifically, this method uses hyperpolarized ¹²⁹Xe MRI to detect blood flow and perfusion changes in the region of interest. In addition, this method can be used to detect blood flow changes in brain tissue that corresponds to the brain functional activities by detecting the amount of ¹²⁹Xe dissolved in blood and brain tissue per unit of time.

### PRIOR APPLICATION INFORMATION

The instant application claims the benefit of U.S. Provisional Patent Application Ser. No. 63/062,640, filed Aug. 7, 2020, entitled “A method to detect perfusion and brain functional activities using hyperpolarized ¹²⁹Xe MRI”, the entire contents of which are incorporated herein by reference.

### BACKGROUND OF THE INVENTION

Methods known in the art for detecting perfusion include:

Arterial Spin Labeling (ASL) MRI. The ASL technique quantifies the perfusion of the organ of interest by applying spin tagging to the blood before it enters the region of interest. Once the tagged blood flows through the imaging region, a signal decrease is observed and by subtracting the tagged image from the reference image, the perfusing image is created. While ASL imaging is a widely used clinical method to quantify tissue perfusion, ASL images usually are low-contrast with poor signal-to-noise ratio due to the small signal difference between the control image and the tagged image.

Dynamic Contrast Enhanced (DCE) MRI. DCE is the most widely used clinical method for measuring tissue perfusion and relies on injection of gadolinium (Gd)-based contrast agents and computation of arterial input function. Although DCE MRI is a gold-standard for perfusion imaging, the Gd agents are not capable of crossing the blood-brain barrier meaning that this technique is inapplicable for cerebral perfusion imaging. In addition to Gd agents being slightly toxic, allergic reactions to Gd-based agents are possible and might cause complications during perfusion imaging of a patient allergic to Gd.

Methods known in the art for detecting brain function activities include:

Blood Oxygen Level Depend (BOLD) functional MRI. The BOLD technique detects the changes of paramagnetic deoxyhemoglobin to diamagnetic oxyhemoglobin concentration that take place with brain activation and result in a decreased signal detectable by MRI. While fMRI has demonstrated good correlation of results when compared with PET and EEG, this technique requires sophisticated statistical analysis methods to interpret the results, due to the small signal differences it captures.

Functional Magnetic Resonance Imaging (fMRI) Methodology Using Transverse Relaxation Preparation and Non-Echo-Planar Imaging (EPI) Pulse Sequences. This method is described in published US Patent Application (US 20160113501 A1). Specifically, described therein is an acquisition scheme for T2-weighted BOLD fMRI. It employs a T2 preparation module to induce the BOLD contrast, followed by a single-shot 3D fast gradient echo (GRE) readout with short echo time (TE<2 ms). The separation of BOLD contrast generation from the readout substantially reduces the “dead time” due to long TE required in spin echo (SE) BOLD sequences. This approach, called “3D T2prep-GRE”, can be implemented with any magnetic resonance imaging machine. This approach is expected to be useful for ultra-high field fMRI studies that require whole brain coverage, or for focusing on regions near air cavities. The concept of using T2 preparation to generate BOLD contrast can be combined with many other fast imaging sequences at any field strength.

System and method for tracking cerebral blood flood flow in fmri. This method is described in published PCT Application WO 2015070046 A1. Described therein is a system and method for analyzing blood flow in a subject's brain. In some embodiments, the method includes analyzing fMRI data to identify signals related to blood flow, and selecting a zero time lag seed regressor using the identified signals. The method also includes correlating the selected seed regressor to identify a subset of the fMRI data that correlates with the seed regressor and is offset in time, combining the subset of the data to determine a time-delayed regressor, and performing repetitions to obtain a number of time-delayed regressors, where for each repetition, the seed regressor is adjusted using a previous time-delayed regressor. The method further includes analyzing the data using the time-delayed regressors to determine blood delivery from vessels across the brain, and generating a report. In some embodiments, a second recursive procedure may be performed using an optimized seed regressor obtained from a first recursive procedure.

### SUMMARY OF THE INVENTION

According to a first aspect of the invention, there is provided a method for imaging and/or mapping activation of a region of an individual's brain during stimulation comprising:

a) generating a resting control image of the individual's brain, said individual inhaling a gas comprising ¹²⁹Xe and holding their breath during imaging by:
i) applying a depolarizing pulse to the individual's brain;
ii) after a first time interval, generating a first resting image of the individual's brain;
iii) applying the depolarizing pulse to the individual's brain;
iv) after a second time interval, generating a second resting image of the individual's brain,
v) generating the resting control image of the individual's brain from the the first rest image and the second rest image;

b) generating a stimulation control image of the individual's brain, said individual inhaling the gas comprising ¹²⁹Xe and holding their breath during imaging, by:
i) applying the depolarizing pulse to the individual's brain;
ii) while subjecting the individual to a stimulus, after a first time period, generating a first stimulation image of the individual's brain;
iii) while subjecting the individual to the stimulus, applying the depolarizing pulse to the individual's brain;
iv) while subjecting the individual to the stimulus, after a second time period, generating a second stimulation image of the individual's brain,
v) generating a stimulation control image of the individual's brain from the first stimulation image and the second stimulation image; and

c) generating an activation image of the individual's brain while subjected to the stimulus from the stimulation control image and the resting control image.

As will be appreciated by one of skill in the art, the time delays referred to above as the first time interval and the second time interval for the resting control image, and as the first time period and the second time period for the stimulation control image, can be of arbitrary length until they fit in a tolerable breath-hold duration. Alternatively, in some embodiments, the first time interval and the first time period may be approximately the same duration and the second time interval and the second time period may be approximately the same duration, that is, within 10%, but this is not necessarily a requirement of the invention. As discussed herein, the delay time actually is the source of image contrast and makes hyperpolarized Xe sensitive to the blood flow variations which are then used to detect functional activity.

According to another aspect of the invention, there is provided a method for generating a perfusion image of a body region of interest of an individual comprising:

a) said individual inhaling a gas comprising ¹²⁹Xe and holding their breath during imaging;

b) applying a depolarizing pulse to the body region of interest;

c) after a first time delay interval, generating a first perfusion interval image of the body region of interest;

d) applying the depolarizing pulse to the body region of interest;

e) after a second time delay interval, generating a second perfusion interval image of the body region of interest, and

f) generating a perfusion image of the body region of interest from the first infusion interval image and the second infusion interval image.

### BRIEF DESCRIPTION OF THE DRAWINGS

FIG. 1. Cerebral perfusion images acquired in sagittal and axial projections using HP ¹²⁹Xe Time-of-Flight pulse sequence. A, F) High resolution ¹H T₂-weighted images of the human brain. B-D) Three TOF dynamic images of the brain acquired in axial projections for three different TOF delay times. The signal increase can be seen with increasing TOF delay. G-I) Three TOF dynamic images of the brain acquired in coronal projections. E, J) Perfusion images reconstructed from HP ¹²⁹Xe TOF images.

FIG. 2. Comparison of Xe functional brain mapping to BOLD fMRI of a spiral visual stimulus. A) Task design and the pulse sequence diagram for the Xe TOF experiment. During the control scan, the participant was watching a gray screen. The participant inhaled 1 L of HP ¹²⁹Xe and held their breath for 20 s. Following the control scan, the procedure was repeated, however, instead of the gray screen, a bright rotating spiral was used as a visual stimulus. The Xe TOF pulse sequence was repeated three times with different time delays. B) The Xe functional brain map illustrates areas of faster Xe wash-in. Increased blood flow was detected in the occipital gyrus (“2”), the inferior parietal lobe (“1”), and the frontal pole (“3”). C) Task design for ¹H BOLD fMRI. The periods of rest and stimulus (20 s each) were alternated over the course of a 180 dynamics EPI acquisition. D) 3D BOLD fMRI map overlaid on top of the SPM standard brain. The activated areas indicated with numbers corresponding to the activated areas shown on Xe functional brain map.

FIG. 3. Comparison of the Xe functional brain map to BOLD fMRI of a dotted visual stimulus. A) Task design and pulse sequence diagram of Xe TOF functional magnetic resonance imaging. The imaging procedure was the same as for the spiral visual stimulus. B) The Xe functional brain map illustrates activated brain regions: the occipital gyrus (“1”), superior parietal lobe (“2”), frontal gyrus (“3”), and part of the insula (dark blue). C) Task design for BOLD fMRI. The BOLD fMRI experiment was performed in the same way as the experiment with the spiral visual stimulus. D) 3D BOLD fMRI map overlaid on top of the SPM standard brain. The activated areas correspond to activated areas shown on the Xe functional brain map. The activated brain regions are indicated with numbers which match the labels on the Xe scan.

FIG. 4. Comparison of Xe functional brain map to BOLD fMRI of the motor task (left hand squeezing). A) Task design and pulse sequence for the Xe functional brain map. The image acquisition was conducted similarly to the visual stimuli study. The control scan was acquired when the participant was unstimulated. During the second breath-hold, the participant was squeezing their left hand repeatedly. B) Xe functional brain map of the activated brain areas during left-hand squeezing. We observed signal from the right posterior precentral gyrus, ie the motor cortex (“1”). C) Task design and pulse sequence for BOLD fMRI. BOLD MRI acquisition was the same as for the visual stimuli. D) 3D BOLD fMRI map overlaid on top of the SPM standard brain. The activated brain area corresponds to the right precentral gyrus.

FIG. 5. Estimated signal enhancement of Xe functional brain maps (black) and proton BOLD images (red). The black and white circles correspond to the signal enhancement for each Xe functional brain map and BOLD fMRI, respectively. The mean estimated percent signal enhancement of the Xe functional brain maps was equal to 123.1±59.6%. On the contrary, the mean estimated percent signal enhancement of the BOLD fMRI scans was equal to 1.3±0.3% (p<0.001).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a method to detect perfusion and brain functional activity using Hyperpolarized xenon-129 (¹²⁹Xe) Time-of-Flight (TOF) Magnetic Resonance Imaging (MRI). Specifically, this method uses hyperpolarized ¹²⁹Xe MRI to detect blood flow and perfusion changes in the region of interest. In addition, this method can be used to detect blood flow changes in brain tissue that corresponds to the brain functional activities by detecting the amount of ¹²⁹Xe dissolved in blood and brain tissue per unit of time.

¹²⁹Xe is chemically inert gas, which has been used as an anaesthetic (1). Inhaled Xe undergoes gas exchange in the lungs and dissolves into the blood along with oxygen (2). It is diffused across the whole body and is carried by blood flow, getting into the brain tissues via cerebral blood flow (CBF) (3-5).

Hyperpolarized (HP) Xenon TOF MRI technique uses hyperpolarized ¹²⁹Xe as an imaging contrast agent to obtain images of localized Xe perfusion and blood flow in the imaged areas of the human body.

It was previously shown that brain responses to the external stimulus lead to changes in local cerebral blood flow (CBF) and local blood volume in the responsive areas of the brain (6, 7). Specifically, the amount of Xe which enters the brain tissue is proportional to the blood flow in that area.

Described herein is a method of determining neuronal activities through calculation of difference between Xe wash-in speed in brain image taken during neuron activation/stimulation and a baseline Xe image of the brain taken without activation/stimulation. This is possible due to equality of Xe wash-in speed and blood flow, which is increased in the areas of activation.

The MR signal strength of a given volume element depends on the total magnetization enclosed in the selected element, which is a product of the concentration of nuclei and the excess spin density (polarization). The HP state is a non-equilibrium state which occurs because the net magnetization does not recover after interactions with radiofrequency (RF) pulses. This fact can be employed to perform blood flow or perfusion measurements. After irradiation of a region of interest (ROI) with an RF pulse of enough power to rotate the ¹²⁹Xe magnetization into the plane perpendicular to its initial orientation (90° pulse), ¹²⁹Xe nuclei become depolarized, meaning no signal can be acquired from them during the following measurements. If we consider an incoming flow of dissolved ¹²⁹Xe, the MRI signal from the ROI after a 90° pulse will be determined only by the amount of fresh ¹²⁹Xe nuclei washed-in to the ROI by the blood flow.

Therefore, if the MRI image is acquired following some time delay (recovery time) after an initial 90° pulse, the signal will depend mainly on the regional flow rate. Repeating this procedure using different time delays will yield images of different signal-to-noise ratios (SNRs). The MRI signal will increase with the recovery time increase because more ¹²⁹Xe nuclei will be entering the ROI. The signal will reach a maximum when all the ¹²⁹Xe nuclei that were initially depolarized are subsequently replaced by fresh polarized nuclei. Further increases of the recovery time will not yield any more signal increase.

Based on this principle, it is possible to measure the signal recovery curve. The rate of increase of this curve (the slope of the curve) will be dependent only of the incoming flow rate. Therefore, by acquiring dynamic images of ¹²⁹Xe after selective time-resolved depolarization, it is possible to quantify the regional blood flow and perfusion. Applying this technique for the brain imaging during stimulation and during the rest state, it is possible to measure cerebral blood flow changes associated with the brain activation. As a result, the activation map can be created. We refer to the proposed time-selected depolarization technique for functional imaging as ¹²⁹Xe TOF functional imaging.

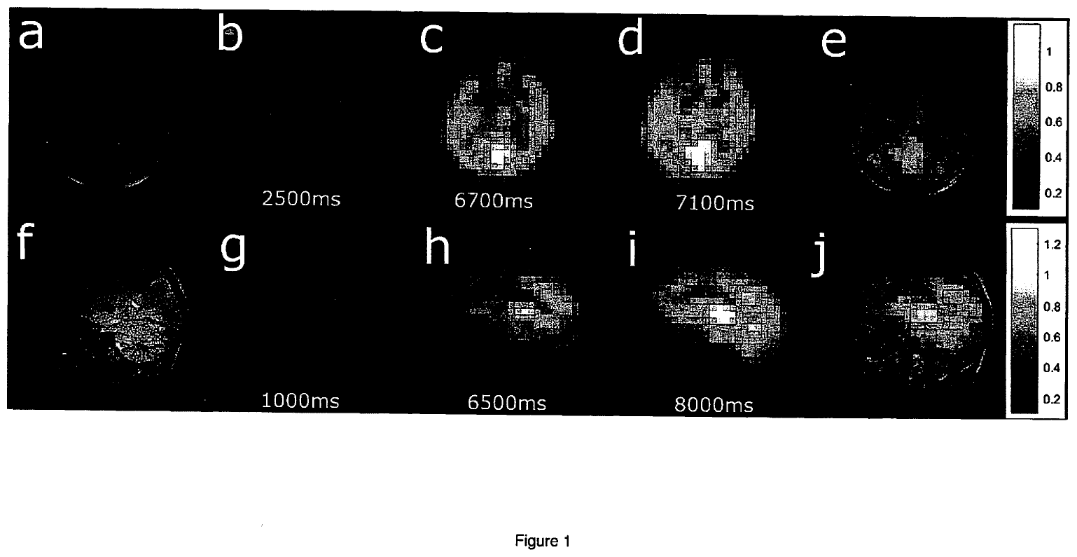

The perfusion images obtained using the HP ¹²⁹Xe TOF MRI technique described herein are shown in FIG. 1. Specifically, three dynamic images were collected while the participant was resting and the net perfusion of the brain tissue was recalculated based on a pixel-by-pixel analysis of the acquired TOF images. The values of the net perfusion acquired using HP ¹²⁹Xe TOF MRI agree well with a known value (8, 9).

The brain activation maps obtained using the HP ¹²⁹Xe TOF MRI technique described herein were compared with the current gold-standard Blood Oxygenation Level Dependent (BOLD) fMRI results. As discussed below, in the HP ¹²⁹Xe TOF MRI method three pairs of dynamic images were acquired with HP ¹²⁹Xe inhalation. Specifically, three dynamic images were collected while the participant was resting, that is, not subjected to any stimulus; subsequently, three dynamic images were taken while the individual was subjected to a specific stimulus, that is, an activity or task.

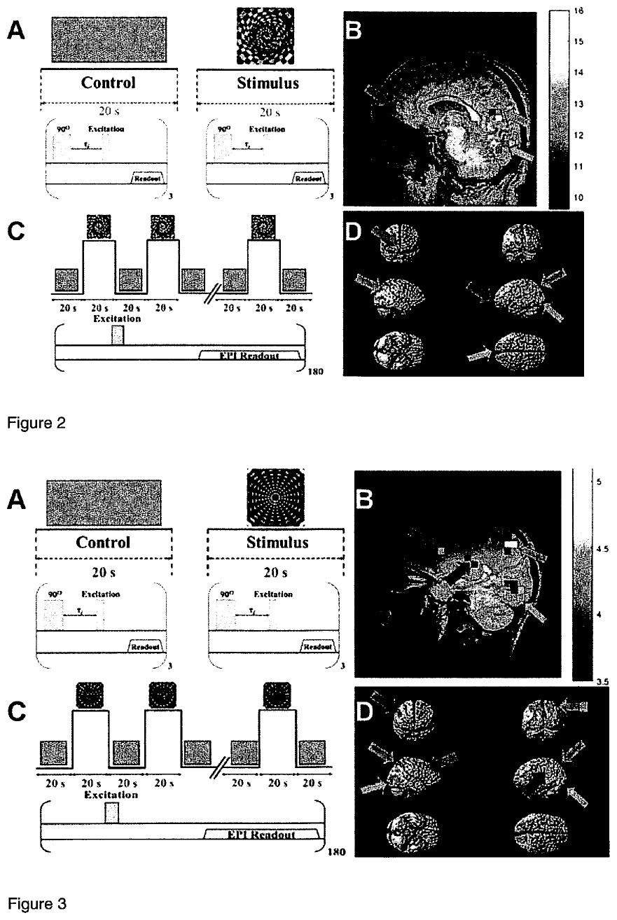

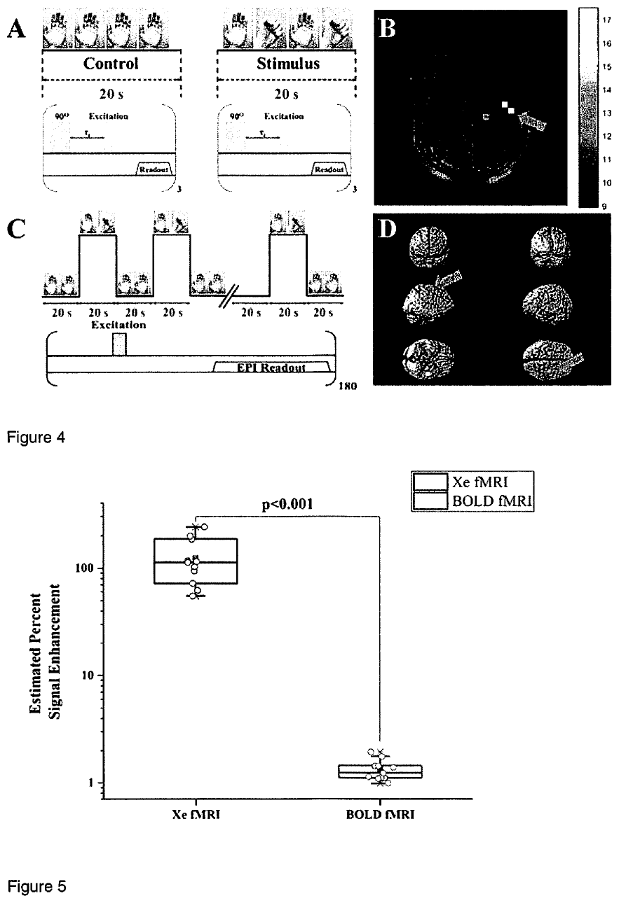

As shown in FIGS. 2-4, the individual was subjected to two visual stimuli: a spiral rotating pattern and a flashing, rotating pattern (FIGS. 2 and 3) and one motor stimulus (left hand clenched and unclenched continuously over the scanning time) (FIG. 4).

As will be appreciated by one of skill in the art, this perfusion imaging technique can be used for quantitative perfusion imaging of highly perfused organs such as for example but by no means limited to the brain, the kidneys, the heart, and the lungs. In addition, HP ¹²⁹Xe TOF pulse sequence can be used to measure and image the blood flow in the cardiovascular system in any part of the body. The signal-to-noise ratio of the acquired images was 10.35 on average which is more than 2 times higher compared to the ASL MRI.

As will be appreciated by one of skill in the art, this method can be used to diagnose of diseases associated with perfusion changes such as for example Alzheimer's disease, Parkinson's disease, multiple renal diseases, atherosclerosis and the like. In addition, this technique can be used for cancer detection since cancerous tumors are characterised by well-developed vasculature, and therefore can be visualized by HP ¹²⁹Xe TOF imaging. The HP ¹²⁹Xe TOF imaging can be used for measuring the pulmonary perfusion and calculation of ventilation/perfusion ratio and, therefore, can be used to diagnostic of pulmonological diseases like asthma, idiopathic pulmonary fibrosis, chronic obstructive pulmonary disease. Moreover, this technique could be used for radiation planning for radiotherapy of lung cancer.

In some embodiments, the imaging procedure is as follows:
a. Participant inhales Xe and holds the breath.
b. At some time point (can be simultaneously with a breath-hold), the image sequence is initiated.
c. Depolarization pulse is applied.
d. Image is taken after a first time period.
e. Depolarization pulse is applied again.
f. Image is taken after a second time period which is different from the first time period.
g. Steps b-f can be repeated multiple times during the same breath-hold using different delay times or time periods.
   Using the acquired multiple images, a perfusion image can be recalculated pixel-by-pixel.

According to a first aspect of the invention, there is provided a method for imaging and/or mapping activation of a region of an individual's brain during stimulation comprising:

a) generating a resting control image of the individual's brain, said individual inhaling a gas comprising ¹²⁹Xe and holding their breath during imaging by:
i) applying a depolarizing pulse to the individual's brain;
ii) after a first time interval, generating a first rest image of the individual's brain;
iii) applying the depolarizing pulse to the individual's brain;
iv) after a second time interval, generating a second rest image of the individual's brain,
v) generating the resting control image of the individual's brain from the the first rest image and the second rest image;

b) generating a stimulation control image of the individual's brain, said individual inhaling the gas comprising ¹²⁹Xe and holding their breath during imaging, by:
i) applying the depolarizing pulse to the individual's brain;
ii) while subjecting the individual to a stimulus, after a first time period, generating a first stimulation image of the individual's brain;
iii) while subjecting the individual to the stimulus, applying the depolarizing pulse to the individual's brain;
iv) while subjecting the individual to the stimulus, after a second time period, generating a second stimulation image of the individual's brain,
v) generating a stimulation control image of the individual's brain from the first stimulation image and the second stimulation image; and

c) generating an activation image of the individual's brain while subjected to the stimulus from the stimulation control image and the resting control image.

In some embodiments of the invention, the stimulus is a visual stimulus, for example, a rotating pattern or a flashing pattern.

In other embodiments of the invention, the stimulus is a mechanical stimulus.

In some embodiments of the invention, the depolarizing pulse is applied at a bandwidth between 1060 and 3533 Hz.

The resting image may be calculated by a pixel-by-pixel comparison of the first resting image and the second resting image.

The stimulation image may be calculated by a pixel-by-pixel comparison of the first stimulation image and the second stimulation image.

The activation image may be calculated by a pixel-by-pixel comparison of the stimulation image and the resting image.

As will be appreciated by one of skill in the art, the time delays referred to above as the first time interval and the second time interval for the resting control image, and as the first time period and the second time period for the stimulation control image, can be of arbitrary length until they fit in a tolerable breath-hold duration. Alternatively, in some embodiments, the first time interval and the first time period may be approximately the same duration and the second time interval and the second time period may be approximately the same duration, that is, within 10%, but this is not necessarily a requirement of the invention. As discussed herein, the delay time actually is the source of image contrast and makes hyperpolarized Xe sensitive to the blood flow variations which are then used to detect functional activity. It is noted that determination of each respective time delay, as defined above, may be determined through routine experimentation, as discussed above.

According to another aspect of the invention, there is provided a method for generating a perfusion image of a body region of interest of an individual comprising:

a) said individual inhaling a gas comprising ¹²⁹Xe and holding their breath during imaging;

b) applying a depolarizing pulse to the body region of interest;

c) after a first time delay interval, generating a first perfusion interval image of the body region of interest;

d) applying the depolarizing pulse to the body region of interest;

e) after a second time delay interval, generating a second perfusion interval image of the body region of interest, and

f) generating a perfusion image of the body region of interest from the first infusion interval image and the second infusion interval image.

As will be appreciated by one of skill in the art, these tasks are examples of motor and visual stimuli, which are the most basic tasks that can be monitored using an fMRI. It is believed that these specific visual stimuli have not been used for fMRI before; however, these visual stimuli each activate multiple brain regions, for example, the regions involved in the visual information recognition, pattern processing and analysis, as discussed herein. These visual tasks were expected to activate the primary and secondary visual cortexes. However, it was hard to predict all activated areas due to the complexity of the stimuli used. As discussed herein, superior parietal lobes were activated as well as frontal gyrus, indicating high involvement of pattern recognition processes.

Furthermore, the motor task activated the motor cortex which is responsible for motion of any part of the body, as discussed herein.

As will be appreciated by one of skill in the art, there were concerns that it would not be possible to detect activation of specific regions of the brain using the instant method. For example, it was possible that the ¹²⁹Xe signal might be too weak due to the low concentration of ¹²⁹Xe in the brain tissue. It was also possible that the regions of the brain indicated as being activated would be different from those detected with the BOLD fMRI method. However, as discussed below, not only were the same regions detected reproducibly, the instant method was able to detect much weaker signals than can be detected with fMRI, as discussed herein.

As will be appreciated by one of skill in the art, this method can be used during brain surgery planning, for example, to determine if the surgery will impact any regions that are activated by various stimuli.

The method can also be used for diagnosis and/or treatment monitoring of different neurodegenerative diseases, for example but by no means limited to Alzheimer's disease and Parkinson disease.

In some embodiments, the imaging procedure is as follows:

1. Taking the resting image:
a. Participant inhales Xe and holds the breath.
b. At some time point (can be simultaneously with a breath-hold), the image sequence is initiated.
c. Depolarization pulse is applied.
d. Image is taken after some delay time.
e. Depolarization pulse is applied again.
f. Image is taken after a delay time which is different from the previously used.
g. Steps b-f can be repeated multiple times during the same breath-hold using different delay times.

2. Taking the stimulated image
a. Participant inhales Xe, holds the breath, and subjects to the stimulus.
b. At some time point (can be simultaneously with a breath-hold), the image sequence is initiated.
c. Depolarization pulse is applied.
d. Image is taken after some delay time.
e. Depolarization pulse is applied again.
f. Image is taken after a delay time which is different from the previously used.
g. Steps b-f can be repeated multiple times during the same breath-hold using different delay times.

As will be appreciated by one of skill in the art, the time intervals can be of any length. However, the net sum of time intervals and imaging time should not exceed the breath-hold duration. Similarly, the difference between the delay times can be of any value until a visible change in image signal is noticed. In many cases, this will depend on the scanner hardware and coil sensitivity.

As will be appreciated by one of skill in the art, the pulse must have a bandwidth broad enough to suppress the all brain signal, but not too broad to touch the gas phase. In some embodiments, the pulse may have a bandwidth between 1060 and 3533 Hz.

The pulse sequence for acquisition includes a depolarizing pulse which is followed by image acquisition after some time delay (TOF time). By varying TOF delay dynamically, it is possible to measure the perfusion of the brain tissue. The signal-to-noise ratio (SNR) was determined for all images and rate of increase was calculated for each set (with stimulus and without). The map of brain activation reflecting the brain functional activities was obtained after subtraction of SNR increase in set without stimulus from the set acquired with stimulus.

Since the HP state is a non-equilibrium metastable state, the longitudinal magnetization is not restored by spin-lattice relaxation once a radiofrequency (RF) pulse irradiates the nuclei. After irradiation of a volume element containing HP ¹²⁹Xe, dissolved in tissue or blood, with a 90° RF pulse, the HP state is completely destroyed and dissolved HP ¹²⁹Xe will not produce any significant amount of signal. If there is continuous flow into the volume of dissolved ¹²⁹Xe, and if the MR measurement is conducted following a prescribed time delay (time-of-flight (TOF) time), the MR signal will be determined mainly by the amount of Xe washed into the selected volume. Due to activation of the brain region, the incoming blood flow into the activated region is significantly increased. Therefore, the speed of incoming ¹²⁹Xe increases as well. By calculating the wash-in rate of Xe during rest and while subjected to stimulus, it is possible to map the difference caused by increased flow into the activated regions. As a result, it is possible to create an activation map showing what regions of the brain are activated by different stimuli, as discussed herein.

The acquired TOF functional images are shown on FIG. 2B, FIG. 3B, and FIG. 4B. Regions of the brain identified with the same numbers on pictures B and D on FIGS. 2, 3 and 4, indicating correspondence between BOLD fMRI and ¹²⁹Xe TOF MRI results. It can be clearly seen that both imaging modalities detected activation of the same brain regions. However, the percent signal enhancement was up to two orders of magnitude higher in ¹²⁹Xe TOF MRI technique than in the conventional BOLD MRI (FIG. 5). The statistical significance of this result has been identified by Student's t-test.

As will be appreciated by one of skill in the art, the superior signal enhancement demonstrates the higher sensitivity of the instant invention compared to BOLD fMRI. This means that HP ¹²⁹Xe TOF can be used for imaging and studying the weaker brain stimuli which were undetectable on BOLD scans. In addition, this technique does not require multiple repetitions of the rest/stimuli imaging for functional image creation. Therefore, HP ¹²⁹Xe TOF functional imaging can be successfully used for studying stimuli which cannot be repeat frequently.

As will be appreciated by one of skill in the art, this technique can be used in a variety of ways, one example of which is to study brain function changes associated with a neurodegenerative diseases or stroke. For example, in a clinical setting, the HP ¹²⁹Xe TOF could be used to study Alzheimer's disease associated memory changes by subjecting the subject to a variety of memory tasks. Specifically, it is possible to study both short-term and long-term memory by slightly changing the activation task during imaging. Other suitable uses will be readily apparent to one of skill in the art.

The invention will now be further explained and/or elucidated by way of examples; however, the invention is not necessarily limited to or by the examples.

### Example 1. Cerebral Perfusion Images of Healthy Volunteer Acquired in Axial and Sagittal Projections

Panels B-D and G-I on FIG. 1 show the dynamic TOF images acquired at three different times. The increase of the SNR with increase of TOF time can be clearly seen. Panels A and F show the high-resolution proton localization of the brain. Panel E and J show the perfusion images with SNR of 9.2 and 11.2 respectively.

### Example 2—Comparison of Xe Functional Brain Mapping to BOLD fMRI of a Spiral Visual Stimulus

The task design and pulse sequence diagram for the Xe TOF experiment are shown generally in Panel A of FIG. 2. As can be seen, during the control scan, the participant was watching a gray screen. The participant inhaled 1 L of HP ¹²⁹Xe and held their breath for 20 s and a brain image was taken. Once the control scan was taken, the procedure was repeated while the participant was shown a bright rotating spiral as a visual stimulus. The Xe TOF pulse sequence was repeated three times, each time following a different time delay.

Panel B of FIG. 2 shows the Xe functional brain map which illustrates areas of faster Xe wash-in, that is, areas of activation. As can be seen in panel B of FIG. 1, increased blood flow was detected in the occipital gyrus, shown by the number “2”, the inferior parietal lobe, shown by the number “1”, and the frontal pole, shown by the number “3”.

Panel C of FIG. 2 shows the task design for ¹H BOLD fMRI. The periods of rest and stimulus (20 s each) were alternated over the course of a 180 dynamics EPI acquisition.

Panel D of FIG. 2 shows the 3D BOLD fMRI map overlaid on top of the SPM standard brain. As can be seen, the activated areas indicated with numbers correspond to the activated areas shown on the Xe functional brain map shown in panel B.

### Example 3—Comparison of the Xe Functional Brain Map to BOLD fMRI of a Dotted Visual Stimulus

Panel A of FIG. 3 shows the task design and pulse sequence diagram of Xe TOF functional magnetic resonance imaging. As can be seen, the imaging procedure was the same as for the spiral visual stimulus.

Panel B of FIG. 3 shows the Xe functional brain map generated, which illustrates the brain regions activated by exposure to a flashing spiral. As can be seen, the occipital gyrus (shown by the number “1”), the superior parietal lobe (shown by the number “2”), the frontal gyrus (shown by the number “3”), and part of the insula.

It is of note that while in both FIGS. 2 and 3, the occipital gyrus showed activation, the inferior parietal lobe and the frontal pole were only active when exposed to the rotating spiral and the superior parietal lobe, the frontal gyrus and part of the insula were only activated by exposure to or stimulus with the flashing spiral pattern.

Panel C of FIG. 3 summarizes the task design for BOLD fMRI. As can be seen, the BOLD fMRI experiment was performed in the same way as the experiment with the spiral visual stimulus.

Panel D of FIG. 3 shows the 3D BOLD fMRI map overlaid on top of the SPM standard brain. The activated areas correspond to activated areas shown on the Xe functional brain map. The activated brain regions are indicated with numbers which match the labels on the Xe scan.

### Example 4—Comparison of Xe Functional Brain Map to BOLD fMRI During the Motor Task (Left Hand Squeezing)

Panel A of FIG. 4 shows the task design and pulse sequence for the Xe functional brain map. The image acquisition was conducted similarly to the visual stimuli study. Specifically, the control scan was acquired when the participant was unstimulated. During the second breath-hold, the participant was squeezing their left hand repeatedly.

Panel B of FIG. 4 shows the Xe functional brain map of the activated brain areas during left-hand squeezing. We observed signal from the right posterior precentral gyrus, ie the motor cortex, labelled with number “1”.

Panel C of FIG. 4 shows the task design and pulse sequence for BOLD fMRI. BOLD MRI acquisition, which was the same as for the visual stimuli.

Panel D of FIG. 4 shows the 3D BOLD fMRI map overlaid on top of the SPM standard brain. The activated brain area corresponds to the right precentral gyrus. As will be appreciated by one of skill in the art, FIGS. 1-3 demonstrate the agreement between Xe TOF and BOLD indicating that results obtained with Xe TOF fMRI are correct. The intensities in panel B of FIGS. 1-3 show the change in the Xe TOF slope. This could be recalculated into the perfusion difference between rest and stimulus.

### Example 5—Estimated Signal Enhancement of Xe Functional Brain Maps and Proton BOLD Images

Referring to FIG. 5, the black and white circles correspond to the signal enhancement for each Xe functional brain map and BOLD fMRI, respectively. The mean estimated percent signal enhancement of the Xe functional brain maps was equal to 123.1±59.6%. On the contrary, the mean estimated percent signal enhancement of the BOLD fMRI scans was equal to 1.3±0.3% (p<0.001).

Compared to Blood Oxygen Level Dependent (BOLD) MRI, the signal to noise ratio and spatial resolution of the result maps generated from acquired images using this technique are clearly higher. Unlike BOLD fMRI, which detects neuroactivities via the changes in deoxyhemoglobin concentration following stimulus, this technique detects the changes in the hemodynamic process (cerebral blood flow), which creates much higher contrasts, and therefore has an enhanced detection ability. In addition, compared to Electroencephalography (EEG), while EEG is superior in temporal resolution, EEG severely lacks spatial information of detected signals, limiting its use to little more than global signal detection. This invention overcomes these limitations of the existing techniques.

While the preferred embodiments of the invention have been described above, it will be recognized and understood that various modifications may be made therein, and the appended claims are intended to cover all such modifications which may fall within the spirit and scope of the invention.

### REFERENCES

1. Albert, M. S. et al. Biological Magnetic Resonance Imaging Using Laser-Polarized 129Xe. Nature vol. 370 199-201 (1994).
2. Chang, Y. V. MOXE: A model of gas exchange for hyperpolarized 129Xe magnetic resonance of the lung. Magn. Reson. Med. 69, 884-890 (2013).
3. Hane, F. T. et al. Inhaled Xenon Washout as a Biomarker of Alzheimer's Disease. Diagnostics 8, E41 (2018).
4. Rao, M., Stewart, N. J., Norquay, G., Griffiths, P. D. & Wild, J. M. High Resolution Spectroscopy and Chemical Shift Imaging of Hyperpolarized 129 Xe Dissolved in the Human Brain In Vivo at 1.5 Tesla. Magn. Reson. Med. 75, 2227-2234 (2016).
5. Rao, M. R., Stewart, N. J., Griffiths, P. D., Norquay, G. & Wild, J. M. Imaging Human Brain Perfusion with Inhaled Hyperpolarized 129 Xe MR Imaging. Radiology 286, 659-665 (2017).
6. Logothetis, N. K. & Pfeuffer, J. On the nature of the BOLD fMRI contrast mechanism. Magn. Reson. Imaging 22, 1517-1531 (2004).
7. Logothetis, N. K. What we can do and what we cannot do with fMRI. Nature 453, 869-878 (2008).
8. Kilian, W., Seifert, F. & Rinneberg, H. Dynamic NMR Spectroscopy of Hyperpolarized 129Xe in Human Brain Analyzed by an Uptake Model. Magn. Reson. Med. 51, 843-847 (2004).
9. Peled, S. et al. Determinants of tissue delivery for 129Xe magnetic resonance in humans. Magn. Reson. Med. 36, 340-344 (1996).

1. A method for imaging and/or mapping activation of a region of an individual's brain during stimulation comprising:
a) generating a resting control image of the individual's brain, said individual inhaling a gas comprising ¹²⁹Xe and holding their breath during imaging by:
i) applying a depolarizing pulse to the individual's brain;
ii) after a first time interval, generating a first resting image of the individual's brain;
iii) applying the depolarizing pulse to the individual's brain;
iv) after a second time interval, generating a second resting image of the individual's brain,
v) generating the resting control image of the individual's brain from the first resting image and the second resting image;
b) generating a stimulation control image of the individual's brain, said individual inhaling the gas comprising ¹²⁹Xe and holding their breath during imaging, by:
i) applying the depolarizing pulse to the individual's brain;
ii) while subjecting the individual to a stimulus, after a first time period, generating a first stimulation image of the individual's brain;
iii) while subjecting the individual to the stimulus, applying the depolarizing pulse to the individual's brain;
iv) while subjecting the individual to the stimulus, after a second time period, generating a second stimulation image of the individual's brain,
v) generating a stimulation control image of the individual's brain from the first stimulation image and the second stimulation image; and
c) generating an activation image of the individual's brain while subjected to the stimulus from the stimulation control image and the resting control image.

2. The method according to claim 1 wherein the stimulus is a visual stimulus.

3. The method according to claim 2 wherein the visual stimulus is a rotating pattern.

4. The method according to claim 2 wherein the visual stimulus is a flashing pattern.

5. The method according to claim 1 wherein the stimulus is a mechanical stimulus.

6. The method according to claim 1 wherein the depolarizing pulse is applied at a bandwidth between 1060 and 3533 Hz.

7. The method according to claim 1 wherein the resting image is calculated by a pixel-by-pixel comparison of the first resting image and the second resting image.

8. The method according to claim 1 wherein the stimulation image is calculated by a pixel-by-pixel comparison of the first stimulation image and the second stimulation image.

9. The method according to claim 1 wherein the activation image is calculated by a pixel-by-pixel comparison of the stimulation image and the resting image.

10. A method for generating a perfusion image of a body region of interest of an individual comprising:
a) said individual inhaling a gas comprising ¹²⁹Xe and holding their breath during imaging;
b) applying a depolarizing pulse to the body region of interest;
c) after a first perfusion time interval, generating a first perfusion interval image of the body region of interest;
d) applying the depolarizing pulse to the body region of interest;
e) after a second perfusion time interval, generating a second perfusion interval image of the body region of interest, and
f) generating a perfusion image of the body region of interest from the first infusion interval image and the second infusion interval image.

11. The method according to claim 10 wherein the depolarizing pulse is applied at a bandwidth between 1060 and 3533 Hz.

12. The method according to claim 10 wherein the body region of interest is an organ.

13. The method according to claim 12 wherein the organ is the brain, the kidney, the lung or the heart.

14. The method according to claim 10 wherein the perfusion image is calculated by a pixel-by-pixel comparison of the first infusion interval image and the second infusion interval image.
